SEQUENCE LISTING (25 sequences)

SEQ ID NO: 1 - protein, 121 aa, Artificial Sequence [sequence omitted]

SEQ ID NO: 2 - protein, 107 aa, Artificial Sequence [sequence omitted]

SEQ ID NO: 3 - protein, 119 aa, Artificial Sequence
  Synthetic
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVS

SEQ ID NO: 4 - protein, 107 aa, Artificial Sequence
  Synthetic
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 5 - protein, 115 aa, Artificial Sequence
  Synthetic
QVQLKESGPGLVAPSQSLSITCTVSGFSLTDYGVNWVRQPPGKGLEWLGMIWGDGSTDYDSTLKSRLSISKDNSKSQIFLKMNSLQTDDTARYYCAREWHHGPVAYWGQGTLTVS

SEQ ID NO: 6 - protein, 107 aa, Artificial Sequence
  Synthetic
DIVMTQSHKFMSTTVGDRVSITCKASQAVSSAVAWYQQKPGQSPKLLIYWASTRHTGVPDRFTGSGSVTDFTLTIHNLQAEDLALYYCQQHYSTPFTFGSGTKLEIK

SEQ ID NO: 7 - protein, 126 aa, Artificial Sequence
  Synthetic
  MISC_FEATURE (28)..(28): Xaa is Ile or Thr.
  MISC_FEATURE (43)..(43): Xaa is Lys or Asn.
  MISC_FEATURE (50)..(50): Xaa is Ile, Phe or Val.
  MISC_FEATURE (51)..(51): Xaa is Ile or Met.
  MISC_FEATURE (52)..(52): Xaa is Ser or Leu
  MISC_FEATURE (53)..(53): Xaa is Tyr or Phe
  MISC_FEATURE (59)..(59): Xaa is Lys or Tyr.
  MISC_FEATURE (60)..(60): Xaa is Ser or Tyr.
  MISC_FEATURE (66)..(66): Xaa is Asp or Gly.
  MISC_FEATURE (70)..(70): Xaa is Val or Ile.
  MISC_FEATURE (75)..(75): Xaa is Ser or Pro.
  MISC_FEATURE (78)..(78): Xaa is Thr or Ala.
  MISC_FEATURE (80)..(80): Xaa is Tyr or Phe.
  MISC_FEATURE (94)..(94): Xaa is Tyr or Phe.
  MISC_FEATURE (102)..(102): Xaa is Ile or Val.
QVQLVESGGGVVQPGRSLRLSCAASGFXFSSYAMHWVRQAPGXGLEWVAXXXXDGSNKXXADSVKXRFTXSRDNXKNXLXLQMNSLRAEDTAVXYCARDRGXSAGGNYYYYGMDVWGQGTTVTVSS

SEQ ID NO: 8 - protein, 108 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 9 - protein, 118 aa, Artificial Sequence
  Synthetic
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSS

SEQ ID NO: 10 - protein, 107 aa, Artificial Sequence
  Synthetic
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIK

SEQ ID NO: 11 - protein, 7 aa, Artificial Sequence
  Synthetic
AMHWVRQ

SEQ ID NO: 12 - protein, 11 aa, Artificial Sequence
  Synthetic
TAVYYCAKVSY

SEQ ID NO: 13 - protein, 11 aa, Artificial Sequence
  Synthetic
ASQGIRNYLAW

SEQ ID NO: 14 - protein, 11 aa, Artificial Sequence
  Synthetic
VATYYCQRYNR

SEQ ID NO: 15 - protein, 13 aa, Artificial Sequence
  Synthetic
SKLTVDKSRWQQG

SEQ ID NO: 16 - protein, 15 aa, Artificial Sequence
  Synthetic
NHWMNWVRQSPEKGL

SEQ ID NO: 17 - protein, 15 aa, Artificial Sequence
  Synthetic
EDTGVYYCSRNYYGS

SEQ ID NO: 18 - protein, 15 aa, Artificial Sequence
  Synthetic
QFVGSSIHWYQQRTN

SEQ ID NO: 19 - protein, 15 aa, Artificial Sequence
  Synthetic
YCQQSHSWPFTFGSG

SEQ ID NO: 20 - protein, 21 aa, Artificial Sequence
  Synthetic
MALPVTALLLPLALLLHAARP

SEQ ID NO: 21 - protein, 21 aa, Artificial Sequence
  Synthetic
IYIWAPLAGTCGVLLLSLVIT

SEQ ID NO: 22 - protein, 45 aa, Artificial Sequence
  Synthetic
TTTPAPRPPTPAPTIASQPLSLRPEACRPAAGGAVHTRGLDFACD

SEQ ID NO: 23 - protein, 42 aa, Artificial Sequence
  Synthetic
KRGRKKLLYIFKQPFMRPVQTTQEEDGCSCRFPEEEEGGCEL

SEQ ID NO: 24 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 15 aa, Artificial Sequence
  Synthetic
GGGGSGGGGSGGGGS

(19) United States
(12) Patent Application Publication
(10) Pub. No.: US 2023/0287097 A1
(43) Pub. Date: Sep. 14, 2023

(54) COMPOSITIONS AND METHODS TO TARGET ANTI-TNF-ALPHA ANTIBODY

(71) Applicant: PORTON BIOLOGICS LTD, Suzhou, Jiangsu (CN)

(72) Inventors: Lingjie KONG, Union City, CA (US); Jean WANG, Union City, CA (US); Tianwei GUO, Suzhou, Jiangsu (CN); Shicheng ZHU, Suzhou, Jiangsu (CN); Ying CHEN, Suzhou, Jiangsu (CN)

(21) Appl. No.: 18/005,669

(22) PCT Filed: Jul. 15, 2021
(86) PCT No.: PCT/CN2021/106404

§ 371 Date: Jan. 16, 2023

(30) Foreign Application Priority Data
Jul. 16, 2020 (WO) PCT/CN2020/102367

(51) Int. Cl. C07K 16/24; C07K 14/705; C07K 14/725; A61K 39/395; A61K 38/17; A61K 39/00

(52) U.S. Cl. CPC C07K 16/241; C07K 14/70517; C07K 14/70578; C07K 14/7051; A61K 39/3955; A61K 38/1774; A61K 38/177; A61K 39/4611; A61K 39/4631; A61K 39/46433; A61K 2039/505; C07K 2319/33; C07K 2319/03; C07K 2319/02; C07K 2317/622; C07K 2317/21; C07K 2317/24; A61K 2239/13; A61K 2239/17; A61K 2239/21; A61K 2239/22

(57) ABSTRACT

Provided a chimeric anti-drug antibody receptor (CADAR) specific for anti-drug-antibody-based B cell receptor (BCR), the anti-drug antibody is induced by a therapeutic anti-TNF-alpha monoclonal antibody. Also provided compositions comprising the CADAR, polynucleotides encoding the CADAR, vectors comprising a polynucleotide encoding the CADAR, engineered cells comprising the CADAR, and method using the same.

### CROSS-REFERENCE TO RELATED APPLICATIONS

This application claims priority PCT application no. PCT/CN2020/102367, filed Jul. 16, 2020, the disclosure of which is incorporated herein by reference.

### FIELD OF THE INVENTION

The present disclosure generally relates to therapeutics including treatment with immunosuppressive medication. In particular, the present disclosure relates to compositions and methods for boosting response to the treatment with a therapeutic anti-TNF alpha monoclonal antibody.

### BACKGROUND

The use of therapeutic monoclonal antibodies in the treatment of cancer, autoimmune diseases and other indications has experienced significant expansion in the recent years. A well-known side effect associated with the therapeutic antibodies is the development of anti-drug antibodies (ADAs), which interfere with therapy outcomes. ADAs can lead to enhanced clearance of the therapeutic antibodies and prevent the drug from binding to the target. Notwithstanding their importance, the molecular landscape of ADAs and the mechanism involved in their formation are not fully understood, much less possible mitigation strategies. Efforts to develop chimeric, humanized and fully human antibodies did not fully abolish the immunogenicity of the therapeutic antibodies and the associated induction of ADAs.

Therapeutic monoclonal antibodies targeting tumor necrosis factor alpha (TNF-alpha) have been widely used in clinics to treat rheumatoid arthritis, inflammatory bowel disease, and other chronic inflammatory associated disorders such as psoriasis, psoriatic arthritis, and ankylosing spondylitis. Currently, at least five anti-TNA-alpha monoclonal antibodies have been approved for various indications. Formation of ADA has been associated with all five agents (van Schouwenburg P A et al. Nat Rev Rheumatol, 2013l 9(3):164, Vaisman-Mentesh A et al., Front. Immunol., 2019; 10:2921). Studies have shown that the presence of ADAs impaired the clinical response to anti-TNA-alpha antibodies and/or elicited adverse events, leading to medical consequences including increase of dosage or dosing frequency, concomitant use of immune modulating drugs, discontinuation of the treatment or switch to other TNF-alpha antagonist (Atiqi S, et al., Front Immunol. 2020; 11:312, Homann A et al. J Transl Med (2015) 13:339). Therefore, a need exists for eliminating the ADAs to boost clinical response and/or eliminate adverse events associated with the therapeutic anti-TNF-alpha monoclonal antibodies.

### SUMMARY OF INVENTION

In one aspect, the present disclosure provides a polynucleotide encoding a chimeric anti-drug antibody receptor (CADAR). In some embodiments, the chimeric anti-drug antibody receptor comprises an extracellular domain comprising an immunogenic fragment of a therapeutic anti-TNF-alpha monoclonal antibody, a transmembrane domain and an intracellular signaling domain, wherein the immunogenic fragment binds to a B cell receptor (BCR) expressed on a B-cell, wherein a cell expressing the CADAR binds the BCR expressed on the B-cell or induces killing of the B-cell expressing the anti-drug antibody.

In some embodiments, the immunogenic fragment comprises a heavy chain variable region or light chain variable region of the therapeutic anti-TNF-alpha monoclonal antibody, a sequence having at least 90% identify thereof, or a sequence having 1, 2, 3, 4, 5 amino acid residue difference therefrom. In some embodiments, the immunogenic fragment comprises a scFV that comprises the heavy chain variable region and the light chain variable region of the therapeutic anti-TNF-alpha monoclonal antibody, a sequence having at least 90% identify thereof, or a sequence having 1, 2, 3, 4, 5 amino acid residue difference therefrom.

In some embodiments, the therapeutic anti-TNF-alpha monoclonal antibody is selected from adalimumab, infliximab, afelimomab, golimumab, and certolizumab. In some embodiments, the immunogenic fragment comprises a heavy chain variable region or light chain variable region as listed in Table 1, a sequence having at least 90% identify thereof, or a sequence having 1, 2, 3, 4, 5 amino acid residue difference therefrom. In some embodiments, the immunogenic fragment comprises a scFv that comprises the paired heavy chain variable region and light chain variable region as listed in Table 1, a sequence having at least 90% identify thereof, or a sequence having 1, 2, 3, 4, 5 amino acid residue difference therefrom.

In some embodiments, the therapeutic anti-TNF-alpha monoclonal antibody is adalimumab and the immunogenic fragment comprises (a) one or more sequences selected from the group of sequences listed in Table 2, or one or more sequences having at least 90% identity thereto, or one or more sequences having 1, 2, 3, 4, or 5 amino acid residue difference therefrom; or (b) a TNF-alpha binding fragment of adalimumab, or a sequence having at least 90% identity thereto, or a sequence having 1, 2, 3, 4, or 5 amino acid residue difference therefrom; or a combination of (a) and (b).

In some embodiments, the therapeutic anti-TNF-alpha monoclonal antibody is infliximab and the immunogenic fragment comprises (a) one or more sequences selected from the group of sequences listed in Table 3, or one or more sequences having at least 90% identity thereto, or one or more sequences having 1, 2, 3, 4, or 5 amino acid residue difference from any of the group of sequences listed in Table 3; or (b) a TNF-alpha binding fragment of infliximab, or a sequence having at least 90% identity thereto, or a sequence having 1, 2, 3, 4, or 5 amino acid residue difference therefrom; or a combination of (a) and (b).

In some embodiments, the chimeric receptor further comprises a signal peptide of CD8 alpha. In some embodiments, the signal domain of CD8 alpha comprises the sequence of SEQ ID NO: 20 or a sequence having at least 90% identity thereto or a sequence having 1, 2, 3, 4, or 5 amino acid residue difference therefrom.

In some embodiments, the transmembrane domain comprises a transmembrane domain of CD8 alpha. In some embodiment, the transmembrane domain of CD8 alpha comprises the sequence of SEQ ID NO: 21, or a sequence having at least 90% identity thereto or a sequence having 1, 2, 3, 4, or 5 amino acid residue difference therefrom.

In some embodiment, the extracellular domain is linked to the transmembrane domain by a hinge region. In some embodiment, the hinge region comprises a hinge region of CD8 alpha. In some embodiment, the hinge region of CD8 alpha comprises the sequence of SEQ ID NO: 22, or a sequence having at least 90% identity thereto or a sequence having 1, 2, 3, 4, or 5 amino acid residue difference therefrom.

In some embodiments, the intracellular domain comprises a costimulatory domain and a signaling domain. In some embodiments, the costimulatory domain comprises an intracellular domain of CD137. In some embodiments, the intracellular domain of CD137 comprises the sequence of SEQ ID NO: 23, or a sequence having at least 95% identity thereto.

In some embodiments, the intracellular domain comprises a signaling domain of CD3 zeta. In some embodiments, the signaling domain of CD3 zeta comprises the sequence of SEQ ID NO: 24, or a sequence having at least 95% identity thereto.

In another aspect, the present disclosure provides a polypeptide encoded by the polynucleotide as describe herein.

In another aspect, the present disclosure provides vector comprising the polynucleotide as described herein, wherein the polynucleotide encoding the CADAR is operatively linked to at least one regulatory polynucleotide element for expression of the CADAR.

In some embodiments, the vector is a plasmid vector, a viral vector, a transposon, a site directed insertion vector, or a suicide expression vector. In some embodiments, the vector is a lentiviral vector, a retroviral vector, or an AAV vector.

In another aspect, the present disclosure provides an engineered cell comprising the vector as described herein.

In some embodiment, the engineered cell is a T cell or an NK cell.

In another aspect, the present disclosure provides a method of boosting response to the treatment with a therapeutic anti-TNF alpha monoclonal antibody in a subject in need thereof, comprising administering an effective amount of the engineered cell as described herein.

In some embodiments, the subject has a condition selected from rheumatoid arthritis (RA), Juvenile idiopathic arthritis (JIA), psoriatic arthritis (PsA), ankylosing spondylitis (AS), adult Crohn's disease (CD), pediatric Crohn's disease, ulcerative colitis (UC), plaque psoriasis (Ps), hidradenitis suppurativa (HS) and uveitis (UV).

In some embodiments, the subject does not respond to or lose initial response to the treatment with the therapeutic anti-TNF alpha monoclonal antibody. In some embodiment, the therapeutic anti-TNF alpha monoclonal antibody induces anti-drug antibodies in the subject.

In some embodiment, the engineered cell is an autologous cell. In some embodiments, the engineered cell is an allogeneic cell.

In some embodiments, the method further comprises administering an agent that increases the efficacy of the engineered cells. In some embodiments, the method further comprises administering an agent that ameliorates a side effect associated with the administration of the engineered cells.

### BRIEF DESCRIPTION OF THE DRAWINGS

The accompanying drawings, which are incorporated herein, form part of the specification. Together with this written description, the drawings further serve to explain the principles of, and to enable a person skilled in the relevant art(s), to make and use the present disclosure.

FIG. 1 illustrates that chimeric anti-drug antibody receptor (CADAR) expressed on engineered T cells target B-cell receptor (BCR) expressed on certain B cells that produce ADA against adalimumab.

FIG. 2 illustrates a schematic diagram of an exemplary CADAR construct.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

The following definitions are provided to assist the reader. Unless otherwise defined, all terms of art, notations and other scientific or medical terms or terminology used herein are intended to have the meanings commonly understood by those of skill in the art. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

“Antigen” refers to a molecule that provokes an immune response. This immune response may be either humoral, or cell-mediated response, or both. The skilled artisan will understand that any macromolecule, including virtually all proteins or peptides, can serve as an antigen. It is readily apparent that the present disclosure includes therapeutic antibodies acting as antigen eliciting immune response.

“Antibody” refers to a polypeptide of the immunoglobulin (Ig) family that binds with an antigen. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen.

“Monoclonal antibody” refers to an antibody that is made by identical immune cells that are all clones of a unique parent cell.

“Anti-idiotypic antibody” refers to an antibody which binds to the idiotype of another antibody.

“Idiotype” refers to the antigenic determinants of immunoglobulin molecules that are located in the variable region of the antibodies.

“Anti-drug antibody” or “ADA” refers to antibodies elicited in vivo by a therapeutic drug, including a therapeutic antibody. ADAs are directed against immunogenic parts of the therapeutic drug and may affect the efficacy, pharmacokinetics and safety of the treatment with the therapeutic antibody.

“Autologous” cells refer to any cells derived from the same subject into which they are later to be re-introduced.

“Allogeneic” cells refer to any cells derived from a different subject of the same species.

“B-cell receptor” or “BCR” refers to a transmembrane immunoglobulin molecule on the surface of B cell that recognize a specific antigen.

“Chimeric anti-drug antibody receptor” or “CADAR” refers to an engineered receptor that is capable of grafting a desired specificity to an anti-drug antibody into immune cells capable of cell-mediated cytotoxicity. Typically, a CADAR is a fusion polypeptide comprises an extracellular domain that introduces the desired specificity, a transmembrane domain and an intracellular domain that transmits a signal to the immune cells when the immune cells bind to the anti-drug antibody or the specific BCR.

“Co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an major histocompatibility complex (MHC) molecule loaded with peptide, mediates a T cell response, including, but not limited to, proliferation, activation, differentiation, and the like.

“Co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

“Effector cells” used in the context of immune cells refers to cells that can be activated to carry out effector functions in response to stimulation. Effector cells may include, without limitation, NK cells, cytotoxic T cells and helper T cells.

“Effective amount” or “therapeutically effective amount” refers to an amount of a cell, composition, formulation or any material as described here effective to achieve a desirable biological result. Such results may include, without limitation, elimination of B cells expressing a specific BCR and the antibodies produced therefrom. “Epitope” or “immunogenic fragment” or “antigenic determinant” refers to a portion of an antigen recognized by an antibody or an antigen-binding fragment thereof. An epitope can be linear or conformational.

Percentage of “identity” or “sequence identity” in the context of polypeptide or polynucleotide is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Operatively linked” refers to a functional relationship between two or more polynucleotide sequences. In the context of a polynucleotide encoding a fusion protein, such as a polypeptide chain of a CADAR of the disclosure, the term means that the two or more polynucleotide sequences are joined such that the amino acid sequences encoded by these segments remain in-frame. In the context of transcriptional or translational regulation, the term refers to the functional relationship of a regulatory sequence to a coding sequence, for example, a promoter in the correct location and orientation to the coding sequence so as to modulate the transcription.

“Immunogenicity” or “immunogenic” refers to the ability of a foreign substance, such as an antigen, to provoke an immune response in the body of a subject. The immunogenic response typically includes both cell-mediated and humoral arms of the immune response. As used in the context of a therapeutic antibody, an “immunogenic fragment” refers to a region of the antibody that elicit the immune response of the host. Such response can lead to the production of anti-drug antibody (ADA) against the therapeutic antibody compromising the therapeutic effects of the treatment.

“Polynucleotide” or “nucleic acid” refers to a chain of nucleotides. As used herein polynucleotides include all polynucleotide sequences which are obtained by any means available in the art, including, without limitation, recombinant means by synthetic means.

“Polypeptide,” and “protein” are used interchangeably, and refer to a chain of amino acid residues covalently linked by peptide bonds. The polypeptides include natural peptides, recombinant peptides, synthetic peptides, or a combination thereof.

“Single-chain Fv antibody” or “scFv” refers to an engineered antibody comprises a light chain variable region fused to a heavy chain variable region directly or via a peptide linker sequence.

“T cell receptor” or “TCR” refers to a protein complex on the surface of T cells that is responsible for recognizing fragments of antigen as peptides bound to WIC molecules.

“Tumor necrosis factor-α” or “TNF-alpha” is a multifunctional pro-inflammatory cytokine secreted predominantly by monocytes or macrophages that has effects on lipid metabolism, coagulation, insulin resistance, and endothelial function. TNF has been implicated in inflammatory diseases, autoimmune diseases, viral, bacterial and parasitic infections, malignancies, and/or neurodegenerative diseases and is a useful target for specific biological therapy.

“Vector” refers to a vehicle into which a polynucleotide may be operably inserted so as to deliver, replicate or express the polynucleotide. A vector may contain a variety of regulatory elements including, without limitation, origin of replication, promoter, transcription initiation sequences, enhancer, selectable marker genes, and reporter genes. A vector may also include materials to aid in its entry into a host cell, including but not limited to a viral particle, a liposome, or ionic or amphiphilic compounds.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

### Chimeric Anti-Drug Antibody Receptor

Therapeutic monoclonal antibodies targeting TNF-alpha have been widely used in clinics to treat rheumatoid arthritis, inflammatory bowel disease, and other chronic inflammatory associated disorders such as psoriasis, psoriatic arthritis, and ankylosing spondylitis. A well-known side effect associated with the therapeutic anti-TNF-alpha antibodies is the development of anti-drug antibodies (ADAs), which leads to enhanced clearance of the therapeutic antibodies and prevent the drug from binding to the target, thus interfering the therapy outcome.

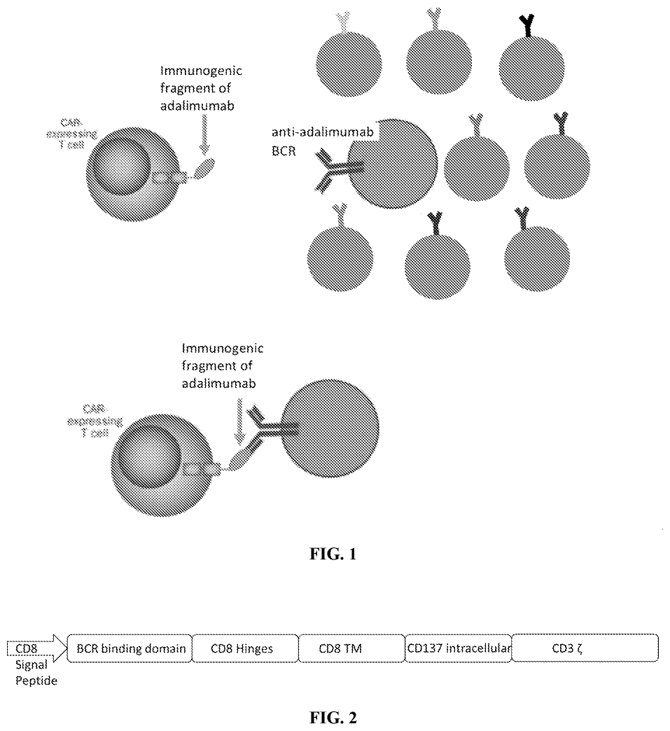

The present disclosure in one aspect relates to the chimeric anti-drug antibody receptors (CADARs) that specifically binds to the B-cell receptor (BCR) expressed on certain B cells that produce ADA against the therapeutic anti-TNF-alpha antibodies (FIG. 1). When the CADARs are expressed on an effector cell, such as a T cell, the CADARs specifically target the effector cells to these B cells, inducing the killing of these B cells, but leaving intact the B cells that do not express and display the ADA against the therapeutic anti-TNF-alpha antibodies. Eliminating the ADA producing B cells improves the treatment efficacy of the therapeutic anti-TNF-alpha antibodies, and alleviates the adverse effects associated with the ADA.

In one aspect, the present disclosure provides a CADAR comprising an extracellular domain, a transmembrane domain and an intracellular signaling domain, whereas the extracellular domain comprises an immunogenic fragment of a therapeutic anti-TNF-alpha monoclonal antibody.

In another aspect, the present disclosure provides a polynucleotide encoding the CADAR described herein.

### Extracellular Domain

In some embodiments, the extracellular domain of the CADAR described herein comprises an immunogenic fragment of a therapeutic anti-TNF-alpha monoclonal antibody. While the immunogenic fragment is recognized by the ADA against the therapeutic anti-TNF-alpha monoclonal antibody, the immunogenic fragment specifically binds to the BCR of the B-cells that express such ADA.

The immunogenic fragment of the present disclosure can be derived from any therapeutic anti-TNF-alpha monoclonal antibodies known in the art, for example, those disclosed in U.S. Pat. Nos. 6,258,562B1, 6,284,471B1, EP2185201A1, U.S. Pat. Nos. 8,241,899B2, 8,603,778B2, 7,521,206B2, 7,012,135B2, 7,186,820B2, 7,402,662B2 and CN1289671C. In some embodiments, the therapeutic anti-TNF-alpha monoclonal antibody from which the immunogenic fragment of the present disclosure is derived is selected from adalimumab, infliximab, afelimomab and golimumab. It should be noted that when reference is made to an anti-TNF-alpha antibody, e.g., adalimumab, the fragments, derivatives and modifications thereof are also included unless the context dictates otherwise.

In some embodiments, the therapeutic anti-TNF-alpha monoclonal antibody from which the immunogenic fragment of the present disclosure is derived comprises the heavy and light chain variable region sequences set forth in Table 1.

TABLE 1
Sequences of exemplary anti-TNF-alpha monoclonal antibodies.

| SEQ | | | |
|---|---|---|---|
| ID NO. | Sequence | Antibody | Domain |
| 1 | EVQLVESGGGLVQPGRSLRLSCAASGFTF | Adalimumab | Heavy chain |
| | DDYAMHWVRQAPGKGLEWVSAITWNSGHI | | variable |
| | DYADSVEGRFTISRDNAKNSLYLQMNSLR | | |
| | AEDTAVYYCAKVSYLSTASSLDYWGQGTL | | region |
| | VTVSS | | |
| 2 | DIQMTQSPSSLSASVGDRVTITCRASQGIR | Adalimumab | Light chain |
| | NYLAWYQQKPGKAPKLLIYAASTLQSGVPS | | variable |
| | RFSGSGSGTDFTLTISSLQPEDVATYYCQR | | region |
| | YNRAPYTFGQGTKVEIK | | |
| 3 | EVKLEESGGGLVQPGGSMKLSCVASGFIFS | Infliximab | Heavy chain |
| | NHWMNWVRQSPEKGLEWVAEIRSKSINSA | | variable |
| | THYAESVKGRFTISRDDSKSAVYLQMTDL | | region |
| | RTEDTGVYYCSRNYYGSTYDYWGQGTTL | | |
| | TVS | | |
| 4 | DILLTQSPAILSVSPGERVSFSCRASQFVG | Infliximab | Light chain |
| | SSIHWYQQRTNGSPRLLIKYASESMSGIPS | | variable |
| | RFSGSGSGTDFTLSINTVESEDIADYYCQQ | | region |
| | SHSWPFTFGSGTNLEVK | | |
| 5 | QVQLKESGPGLVAPSQSLSITCTVSGFSLTD | Afelimomab | Heavy chain |
| | YGVNWVRQPPGKGLEWLGMIWGDGSTDY | | variable |
| | DSTLKSRLSISKDNSKSQIFLKMNSLQTDDT | | region |
| | ARYYCAREWHHGPVAYWGQGTLTVS | | |
| 6 | DIVMTQSHKFMSTTVGDRVSITCKASQAVS | Afelimomab | Light chain |
| | SAVAWYQQKPGQSPKLLIYWASTRHTGVP | | variable |
| | DRFTGSGSVTDFTLTIHNLQAEDLALYYCQ | | region |
| | QHYSTPFTFGSGTKLEIK | | |
| 7 | QVQLVESGGGVVQPGRSLRLSCAASGFXFS | Golimumab | Heavy chain |
| | SYAMHWVRQAPGXGLEWVAXXXXDGSN | | variable |
| | KXXADSVKXRFTXSRDNXKNXLXLQMNS | | region |
| | LRAEDTAVXYCARDRGXSAGGNYYYYGM | | |
| | DVWGQGTTVTVSS | | |
| 8 | EIVLTQSPATLSLSPGERATLSCRASQSVSS | Golimumab | Light chain |
| | YLAWYQQKPGQAPRLLIYDASNRATGIPA | | variable |
| | RFSGSGSGTDFTLTISSLEPEDFAVYYCQQR | | region |
| | SNWPPFTFGPGTKVDIK | | |
| 9 | EVOLVESGGGLVQPGGSLRLSCAASGYVF | Certolizumab | Heavy chain |
| | TDYGMNWVRQAPGKGLEWMGWINTYIGE | | variable |
| | PIYADSVKGRFTFSLDTSKSTAYLQMNSLR | | region |
| | AEDTAVYYCARGYRSYAMDYWGQGTLVT | | |
| | VSS | | |
| 10 | DIQMTQSPSSLSASVGDRVTITCKASQNVG | Certolizumab | Light chain |
| | TNVAWYQQKPGKAPKALIYSASFLYSGVP | | variable |
| | YRFSGSGSGTDFTLTISSLQPEDFATYYCQQ | | region |
| | YNIYPLTFGQGTKVEIK | | |

In certain embodiments, the immunogenic fragment of a therapeutic anti-TNF-alpha monoclonal antibody includes an epitope recognized by an ADA against the therapeutic antibody. It has been discovered that the ADAs can be anti-idiotypic antibodies directed against the antigen-binding region of the therapeutic monoclonal antibody and thus prevent binding of the therapeutic antibody to TNF-alpha.

For example, the sequences of the immunogenic fragments in adalimumab have been mapped by Homann A et al (Theranostics, 2017; 7(19): 4699) and van Schouwenburg P A et al. (J Biol Chem. 2014; 289(50):34482). Exemplary immunogenic fragments of adalimumab are illustrated in Table 2.

TABLE 2
Immunogenic fragment of adalimumab.

| SEQ ID NO. | Sequence | Location |
|---|---|---|
| 11 | AMHWVRQ | VH |
| 12 | TAVYYCAKVSY | VH |
| 13 | ASQGIRNYLAW | VL |
| 14 | VATYYCQRYNR | VL |
| 15 | SKLTVDKSRWQQG | Fc |

Similarly, the sequences of the immunogenic fragments in infliximab have been mapped by Homann et al. (J Transl Med (2015) 13:339). Exemplary immunogenic fragments of infliximab are illustrated in Table 3.

TABLE 3
Exemplary immunogenic fragments of infliximumab

| SEQ ID NO. | Sequence | Location |
|---|---|---|
| 16 | NHWMNWVRQSPEKGL | VH |
| 17 | EDTGVYYCSRNYYGS | VH |
| 18 | QFVGSSIHWYQQRTN | VL |
| 19 | YCQQSHSWPFTFGSG | VL |

In some embodiments, the therapeutic anti-TNF-alpha monoclonal antibody is adalimumab, and the extracellular domain of the CADAR comprises one or more sequences selected from the group of sequences listed in Table 2, or one or more sequences having at least 80%, 85%, 90%, 91%, 92%, 93%, 94%, 95%, 96%, 97%, 98%, 99% sequence identity thereto, or one or more sequences having 1, 2, 3, 4, or 5 amino acid residue difference therefrom.

In some embodiment, the therapeutic anti-TNF-alpha monoclonal antibody is infliximab and the extracellular domain of the CADAR comprises one or more sequences selected from the group of sequences listed in Table 3, or one or more sequences having at least 80%, 85%, 90%, 91%, 92%, 93%, 94%, 95%, 96%, 97%, 98%, 99% sequence identity thereto, or one or more sequences having 1, 2, 3, 4, or 5 amino acid residue difference therefrom.

In some embodiments, the extracellular domain of the CADAR comprises one or more antigen binding fragment of the therapeutic anti-TNF-alpha monoclonal antibody. “Antigen binding fragment” as used herein refers to a portion of an antibody comprising one or more CDRs, or any other antibody fragment that binds to an antigen but does not comprise an intact native antibody structure. It can be understood that the antigen binding fragment in the context of anti-TNF-alpha monoclonal refers to a portion of the antibody that binds to TNF-alpha. Antigen binding fragments useful for the present disclosure include, without limitation, a scFv or a fragment thereof (e.g., VL, VH, CDRs). In some embodiments, the antigen binding fragment is a scFv derived the anti-TNF antibodies listed in Table 1. In some embodiments, the scFv comprises the paired heavy chain variable region and light chain variable region as listed in Table 1.

In some embodiments, the therapeutic anti-TNF-alpha monoclonal antibody is adalimumab, and the extracellular domain of the CADAR comprises a combination of (a) one or more sequences selected from the group of sequences listed in Table 2 or a sequence having at least 80%, 85%, 90%, 91%, 92%, 93%, 94%, 95%, 96%, 97%, 98%, 99% sequence identity thereto, or one or more sequences having 1, 2, 3, 4, or 5 amino acid residue difference therefrom; and (b) an antigen binding fragment of adalimumab, or a sequence having at least 80%, 85%, 90%, 91%, 92%, 93%, 94%, 95%, 96%, 97%, 98%, 99% sequence identity thereto, or one or more sequences having 1, 2, 3, 4, or 5 amino acid residue difference therefrom.

In some embodiments, the therapeutic anti-TNF-alpha monoclonal antibody is infliximab, and the extracellular domain of the CADAR comprises a combination of (a) one or more sequences selected from the group of sequences listed in Table 3 or sequence having at least 80%, 85%, 90%, 91%, 92%, 93%, 94%, 95%, 96%, 97%, 98%, 99% sequence identity thereto, or one or more sequences having 1, 2, 3, 4, or 5 amino acid residue difference therefrom; and (b) an antigen binding fragment of infliximab, or a sequence having at least 80%, 85%, 90%, 91%, 92%, 93%, 94%, 95%, 96%, 97%, 98%, 99% sequence identity thereto, or one or more sequences having 1, 2, 3, 4, or 5 amino acid residue difference therefrom.

In some embodiments, the extracellular domain further comprises a signal peptide. The term “signal peptide” as used herein refers to peptide, usually having a length of 5-30 amino acid residues, present at the N-terminus of a polypeptide that necessary for the translocation cross the membrane on the secretory pathway and control of the entry of the polypeptide to the secretory pathway.

In some embodiments, the signal peptide comprises a signal peptide of CD8 alpha: In some embodiments, the signal peptide of CD8 alpha comprises a sequence of SEQ ID NO: 20 or a sequence having at least 80%, 85%, 90%, 91%, 92%, 93%, 94%, 95%, 96%, 97%, 98%, 99% sequence identity thereto. In some embodiments, the signal peptide comprises a signal peptide of IgG.

### Transmembrane Domain

The transmembrane domain of the CADAR described herein may be derived from any membrane-bound or transmembrane protein including, but are not limited to, BAFFR, BLAME (SLAMF8), CD2, CD3 epsilon, CD4, CD5, CD8, CD9, CD11a (CD18, ITGAL, LFA-1), CD11b, CD11c, CD11d, CD16, CD19, CD22, CD27, CD28, CD29, CD33, CD37, CD40, CD45, CD49a, CD49d, CD49f, CD64, CD80, CD84, CD86, CD96 (Tactile), CD100 (SEMA4D), CD103, CD134, CD137 (4-1BB), CD150 (IPO-3, SLAMF1, SLAM), CD154, CD160 (BY55), CD162 (SELPLG), CD226 (DNAM1), CD229 (Ly9), CD244 (2B4, SLAMF4), CD278 (ICOS), CEACAM1, CRT AM, GITR, HYEM (LIGHTR), IA4, IL2R beta, IL2R gamma, IL7R a, ITGA1, ITGA4, ITGA6, ITGAD, ITGAE, ITGAM, ITGAX, ITGB1, ITGB2, ITGB7, KIR, LTBR, OX40, NKG2C, NKG2D, NKp30, NKp44, NKp46, NKp80 (KLRF1), PAG/Cbp, PSGL1, SLAMF6 (NTB-A, Ly108), SLAMF7, an alpha, beta or zeta chain of a T-cell receptor, TNFR2, VLA1, and VLA-6.

In one embodiment, the CADAR described herein comprises a transmembrane domain of CD8 alpha, CD28 or ICOS. In certain embodiments, the transmembrane domain of CD8 alpha has a sequence of SEQ ID NO: 21, or a sequence having at least 80%, 85%, 90%, 91%, 92%, 93%, 94%, 95%, 96%, 97%, 98%, 99% sequence identity thereto.

In certain embodiments, the transmembrane domain of the CADAR described herein is synthetic, e.g., comprising predominantly hydrophobic residues such as leucine and valine. In certain embodiment, the transmembrane domain of the CADAR described herein is modified or designed to avoid binding to the transmembrane domains of the same or different surface membrane proteins in order to minimize interactions with other members of the receptor complex.

In some embodiments, the CADAR described herein further comprises a hinge region, which forms the linkage between the extracellular domain and transmembrane domain of the CADAR. The hinge and/or transmembrane domain provides cell surface presentation of the extracellular domain of the CADAR.

The hinge region may be derived from any membrane-bound or transmembrane protein including, but are not limited to, BAFFR, BLAME (SLAMF8), CD2, CD3 epsilon, CD4, CD5, CD8, CD9, CD11a (CD18, ITGAL, LFA-1), CD11b, CD11c, CD11d, CD16, CD19, CD22, CD27, CD28, CD29, CD33, CD37, CD40, CD45, CD49a, CD49d, CD49f, CD64, CD80, CD84, CD86, CD96 (Tactile), CD100 (SEMA4D), CD103, CD134, CD137 (4-1BB), CD150 (IPO-3, SLAMF1, SLAM), CD154, CD160 (BY55), CD162 (SELPLG), CD226 (DNAM1), CD229 (Ly9), CD244 (2B4, SLAMF4), CD278 (ICOS), CEACAM1, CRT AM, GITR, HYEM (LIGHTR), IA4, IL2R beta, IL2R gamma, IL7Ra, ITGA1, ITGA4, ITGA6, ITGAD, ITGAE, ITGAM, ITGAX, ITGB1, ITGB2, ITGB7, KIR, LTBR, OX40, NKG2C, NKG2D, NKp30, NKp44, NKp46, NKp80 (KLRF1), PAG/Cbp, PSGL1, SLAMF6 (NTB-A, Ly108), SLAMF7, an alpha, beta or zeta chain of a T-cell receptor, TNFR2, VLA1, and VLA-6.

In some embodiments, the hinge region comprises a hinge region of CD8 alpha, a hinge region of human immunoglobulin (Ig), or a glycine-serine rich sequence.

In some embodiments, the CADAR comprises a hinge region of CD8 alpha, CD28, ICOS or IgG4m. In certain embodiments, the hinge region has a sequence of SEQ ID NO: 22, or a sequence having at least 80%, 85%, 90%, 91%, 92%, 93%, 94%, 95%, 96%, 97%, 98%, 99% sequence identity thereto.

### Intracellular Domain

The intracellular domain of the CADAR described herein, is responsible for activation of at least one of the normal effector functions of the immune cell in which the CADAR has been placed in. The term “effector function” used in the context of an immune cell refers to a specialized function of the cell, for example, the cytolytic activity or helper activity including the secretion of cytokines for a T cell.

It is well recognized that the full activation of a T-cell requires signals generated through the T-cell receptor (TCR) and a secondary or co-stimulatory signal. Thus, the T cell activation is mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

The intracellular domain of the CADAR can be derived from a molecule which transduces the effector function signal and directs the cell to perform the effector function, or a truncated portion of such molecule as long as it transduces the signal. Such protein molecule including, but are not limited to, B7-H3, BAFFR, BLAME (SLAMF8), CD2, CD3 delta, CD3 epsilon, CD3 gamma, CD3 zeta, CD4, CD5, CD7, CD8alpha, CD8beta, CD11a (CD18, LFA-1, ITGAL), CD11b, CD11c, CD11d, CD19, CD27, CD28, CD29, CD30, CD40, CD49a, CD49d, CD49f, CD69, CD79a, CD79b, CD83, CD84, CD86, CD96 (Tactile), CD100 (SEMA4D), CD103, CD127, CD137 (4-1BB), CD150 (SLAM, SLAMF1, IPO-3), CD160 (BY55), CD162 (SELPLG), CD226 (DNAM1), CD229 (Ly9), CD244 (SLAMF4, 2B4), CEACAM1, CRTAM, DAP10, DAP12, common FcR gamma, FcR beta (Fc Epsilon Rib), Fcgamma RIIa, GADS, GITR, HVEM (LIGHTR), IA4, IL2R beta, IL2R gamma, IL7R alpha, ITGA4, ITGA6, ITGAD, ITGAE, ITGAM, ITGAX, ITGB1, ITGB2, ITGB7, ICAM-1, ICOS, LIGHT, LTBR, LAT, NKG2C, NKG2D, NKp44, NKp30, NKp46, NKp80 (KLRF1), OX40, PD-1, PAG/Cbp, PSGL1, SLP-76, SLAMF6 (NTB-A, Ly108), SLAMF7, T cell receptor (TCR), TNFR2, TRANCE/RANKL, VLA1, VLA-6, any derivative, variant, or fragment thereof, any synthetic sequence of a molecule that has the same functional capability, and any combination thereof.

In some embodiments, the intracellular domain comprises a co-stimulatory domain and a signaling domain, wherein upon binding of the CADAR to the ADA, the co-stimulatory domain provides co-stimulatory intracellular signaling without the need of its original ligand, and the signaling domain provides the primary activation signaling. The co-stimulatory domain and the signaling domain of the CADAR can be linked to each other in a random or specified order.

### Co-Stimulatory Domain

In some embodiments, the co-stimulatory domain is derived from an intracellular domain of a co-stimulatory molecule.

Examples of co-stimulatory molecules include B7-H3, BAFFR, BLAME (SLAMF8), CD2, CD4, CD8 alpha, CD8 beta, CD7, CD11a, CD11b, CD11c, CD11d, CD 18, CD 19, CD27, CD28, CD29, CD30, CD40, CD49a, CD49D, CD49f, CD69, CD83, CD84, CD96 (Tactile), CD100 (SEMA4D), CD103, CD 127, CD137(4-1BB), CD150 (SLAM, SLAMF1, IPO-3), CD160 (BY55), CD162 (SELPLG), CD226 (DNAM1), CD229 (Ly9), CD244 (SLAMF4, 2B4), CEACAM1, CRTAM, CDS, OX40, PD-1, ICOS, GADS, GITR, HVEM (LIGHTR), IA4, ICAM-1, IL2R beta, IL2R gamma, IL7R alpha, ITGA4, ITGA4, ITGA6, ITGAD, ITGAE, ITGAL, ITGAM, ITGAX, ITGB1, ITGB2, ITGB7, LAT, LFA-1, LIGHT, LTBR, NKG2C, NKG2D, NKp44, NKp30, NKp46, NKp80 (KLRF1), PAG/Cbp, PSGL1, SLAMF6 (NTB-A, Ly108), SLAMF7, SLP-76, TNFR2, TRANCE/RANKL, VLA1, VLA-6, any derivative, variant, or fragment thereof, any synthetic sequence of a co-stimulatory molecule that has the same functional capability, and any combination thereof.

In some embodiment, the co-stimulatory domain of the CADAR comprises an intracellular domain of co-stimulatory molecule CD137 (4-1BB), CD28, OX40 or ICOS. In some embodiments, the co-stimulatory domain of the CADAR has a sequence of SEQ ID NO: 23. or a sequence having at least 80%, 85%, 90%, 91%, 92%, 93%, 94%, 95%, 96%, 97%, 98%, 99% sequence identity thereto.

### Signaling Domain

The primary activation of the TCR complex can be regulated by a primary cytoplasmic signaling sequence either in a stimulatory manner or in an inhibitory manner. Primary cytoplasmic signaling sequences that act in a stimulatory manner may contain signaling motifs known as immunoreceptor tyrosine-based activation motifs (ITAMs). Examples of ITAM containing primary signaling sequences that are of particular use in the disclosure include those derived from CD3 gamma, CD3 delta, CD3 epsilon, CD3 zata, CD5, CD22, CD79a, CD79b, CD66d, FcR gamma, FcR beta, and TCR zeta.

In some embodiments, the signaling domain of the CADAR of the disclosure comprises an ITAM that provides stimulatory intracellular signaling upon the CADAR binding to the ADA, without HLA restriction. In some embodiments, the signaling domain of the CADAR comprises a signaling domain of CD3 zeta (CD247). In some embodiments, the signaling domain of the CADAR comprises a sequence of SEQ ID NO: 24, or a sequence having at least 80%, 85%, 90%, 91%, 92%, 93%, 94%, 95%, 96%, 97%, 98%, 99% sequence identity thereto.

### Other Region

In some embodiments, the CADAR further comprises a linker. The term “linker” as provided herein is a polypeptide connecting various components of the CADAR.

In some embodiment, the linker is inserted between the VH and VL of the scFv. In some embodiments, the linker is inserted between the transmembrane domain and the intracellular domain. In some embodiments, the linker is between the signaling domain and the co-stimulatory domain of the intracellular domain.

In some embodiments, the linker comprises a glycine-serine (GS) doublet between 2 and 20 amino acid residues in length. Exemplary GS doublets include (G4S)₃: SEQ ID NO: 25. In some embodiments, the polynucleotide provided herein comprises a nucleotide sequence encoding a linker.

In some embodiments, the CADAR provided herein comprises from the N-terminus to the C-terminus: a signal peptide of CD8 alpha, an immunogenic fragment of adalimumab (e.g., a sequence selected from Table 2 or scFv derived from adalimumab), a hinge region of CD8 alpha, a transmembrane domain of CD8 alpha, an intracellular domain of CD137, and a signaling domain of CD3 zeta.

In some embodiments, the polynucleotide provided herein encodes a CADAR comprising from the N-terminus to the C-terminus: a signal peptide of CD8 alpha, an immunogenic fragment of adalimumab (e.g., a scFv derived from adalimumab), a hinge region of CD8 alpha, a transmembrane domain of CD8 alpha, an intracellular domain of CD137, and a signaling domain of CD3 zeta.

In some embodiments, the CADAR demonstrates a high affinity to an ADA against a therapeutic TNF-alpha monoclonal antibody. The term “affinity” as used herein refers to the strength of non-covalent interaction between an immunoglobulin molecule or fragment thereof and an antigen. The binding affinity can be represented by Kd value, i.e., the dissociation constant, determined by any methods known in the art, including, without limitation, enzyme-linked immunosorbent assays (ELISA), surface plasmon resonance, or flow cytometry (such as FACS). In certain embodiments, the CADAR has a binding affinity to the ADA of less than 50 nM, 25 nM, 10 nM, 5 nM, 4 nM, 3 nM, 2 nM, or 1 nM.

### Vector

In another aspect, the present disclosure provides a vector comprising the polynucleotide encoding the CADAR as described herein. The polynucleotides encoding a CAR can be inserted into different types of vectors known in the art, for example, a plasmid, a phagemid, a phage derivative, a viral vector derived from animal virus, a cosmid, transposon, a site directed insertion vector (e.g., CRISPR, Zinc finger nucleases, TALEN), or a suicide expression vector. In some embodiments, the vector is a DNA or RNA.

In some embodiment, the polynucleotide is operatively linked to at least one regulatory polynucleotide element in the vector for expression of the CADAR. Typical vectors contain various regulatory polynucleotide elements, for example, elements (e.g., transcription and translation terminators, initiation sequences, and promoters) regulating the expression of the inserted polynucleotides, elements (e.g., origin of replication) regulating the replication of the vector in a host cell, and elements (e.g., terminal repeat sequence of a transposon) regulating the integration of the vector into a host genome. The expression of the CADAR can be achieved by operably linking the polynucleotides encoding a CADAR to a promoter, and incorporating the construct into a vector. Both constitutive promoters (such as a CMV promoter, a SV40 promoter, and a MMTV promoter), or inducible promoters (such as a metallothionine promoter, a glucocorticoid promoter, and a progesterone promoter) are contemplated for the disclosure. In some embodiment, the vector is an expression vector, An expression vector comprises sufficient cis-acting elements for expression; other elements for expression can be supplied by the host cell or in an in vitro expression system.

In order to assess the expression of a CADAR, the vector can also comprise a selectable marker gene or a reporter gene or both for identification and selection of the cells to which the vector are introduced. Useful selectable markers include, for example, antibiotic-resistance genes, such as neo and the like. Useful reporters include, for example, luciferase, beta-galactosidase, chloramphenicol acetyl transferase, secreted alkaline phosphatase, or the green fluorescent protein gene.

In some embodiments, the vector is a viral vector. Viral vectors may be derived from, for example, retroviruses, adenoviruses, adeno-associated viruses (AAV), herpes viruses, and lentiviruses. Useful viral vectors generally contain an origin of replication functional in at least one organism, a promoter, restriction endonuclease sites, and one or more selectable markers. In some embodiments, the vector is a retrovirus vector, such as lentiviral vector. Lentiviral vector is particular useful for long-term, stable integration of the polynucleotide encoding the CADAR into the genome of non-proliferating cells that result in stable expression of the CADAR in the host cell, e.g., host T cell.

In some embodiments, the vector is mRNA, which can be electroporated into the host cell. As the mRNA would dilute out with cell division, the expression of the mRNA would not be permanent.

In some embodiments, the vector is a transposon-based expression vector. A transposon is a DNA sequence that can change its position within a genome. In a transposon system, the polynucleotide encoding the CADAR is flanked by terminal repeat sequences recognizable by a transposase which mediates the movement of the transposon. A transposase can be co-delivered as a protein, encoded on the same vector as the CADAR, or encoded on a separate vector. Non-limiting examples of transposon systems include Sleeping Beauty, Piggyback, Frog Prince, and Prince Charming.

A vector can be introduced into a host cell, e.g., mammalian cell by any method known in the art, for example, by physical, chemical or biological means. Physical methods for introducing a polynucleotide into a host cell include calcium phosphate precipitation, lipofection, particle bombardment, microinjection, electroporation, and the like. Biological methods include the use of viral vectors, and especially retroviral vectors, for inserting genes into mammalian, e.g., human cells. Chemical means include colloidal dispersion systems, such as macromolecule complexes, nanocapsules, microspheres, beads, and lipid-based systems including oil-in-water emulsions, micelles, mixed micelles, and liposomes.

### Cells

In one aspect, the disclosure provides an engineered cell comprising or expressing the CADAR as described here. In some embodiments, the engineered cell comprises the polynucleotide encoding the CADAR, or the vector comprising the CADAR polynucleotide. In some embodiments, an engineered cell comprises multiple CADAR comprising different immunogenic fragments of a therapeutic anti-TNF-alpha monoclonal antibody.

An engineered cell as described herein is a genetically modified immune cell, Immune cells useful for the disclosure include T cells, natural killer (NK) cells, invariant NK cells, or NKT cells, and other effector cell. In some embodiment, the immune cells are primary cells, expanded cells derived from primary cells, or cells derived from stem cells differentiated in vitro.

It is useful for an engineered cell comprising or expressing a CADAR to have high affinity for ADA-based B cell receptors (BCRs) on B cells, wherein the ADA specifically binds a therapeutic TNF-alpha monoclonal antibody. As a result, the engineered cell can induce direct killing of anti-therapeutic TNF-alpha monoclonal antibody B cells or indirect killing of plasma cells expressing ADA against the therapeutic antibody. In some embodiments, the engineered cell has low affinity for ADA bound to an Fc receptor.

In another aspect, the disclosure provides a method of making an engineered cell expressing the CADAR as described herein. In some embodiments, the method comprising one of more steps selected from of obtaining cells from a source, culturing cells, activating cells, expanding cells and engineering cells

In another aspect, the disclosure provides a method of using the engineered cells for cell therapy, wherein the engineered cells are introducing into a subject. In some embodiments, the subject is the same subject from who the cells are obtained.

### Sources of Cells

The engineered cells can be derived from immune cells isolated from subjects, e.g., human subjects. In some embodiments, the immune cells are obtained from a subject of interest, such as a subject suspected of having a particular disease or condition, a subject suspected of having a predisposition to a particular disease or condition, a subject who will undergo, is undergoing, or have undergone treatment for a particular disease or condition, a subject who is a healthy volunteer or healthy donor, or from blood bank. Thus, the cells can be autologous or allogeneic to the subject of interest. Allogeneic donor cells may not be human-leukocyte-antigen (HLA)-compatible, and thus allogeneic cells can be treated to reduce immunogenicity.

Immune cells can be collected from any location in which they reside in the subject including, but not limited to, blood, cord blood, spleen, thymus, lymph nodes, pleural effusion, spleen tissue, and bone marrow. The isolated immune cells may be used directly, or they can be stored for a period of time, such as by freezing.

In some embodiments, the engineered cells are derived from T cells. T cells can be obtained from blood collected from a subject using any number of techniques known to the skilled artisan, such as apheresis.

In some embodiments, one or more of the T cell populations is enriched for or depleted of cells that are positive for a specific marker, such as surface markers, or that are negative for a specific marker. Such markers are those that are absent or expressed at relatively low levels on certain populations of T cells but are present or expressed at relatively higher levels on certain other populations of T cells. In some embodiments, CD4+ helper and CD8+ cytotoxic T cells are isolated. In some embodiments, CD8+ and CD4+ T cells are further enriched for or depleted of naive, central memory, effector memory, and/or central memory stem cells, such as by positive or negative selection based on surface antigens associated with the respective subpopulation.

### Activation and Expansion of Cells

In some embodiments, the immune cells are activated and expanded prior to genetic modification. In other embodiments, the immune cells are activated, but not expanded, or are neither activated nor expanded prior to use.

Method for activation and expansion of immune cells have been described in the art and can be used in the methods described herein. For example, the T cells can be activated and expanded by contacting with a surface having attached thereto an agent that stimulates a CD3/TCR complex associated signal and a ligand that stimulates a co-stimulatory molecule on the surface of the T cells. To stimulate proliferation of either CD4+ T cells or CD8+ T cells, an anti-CD3 antibody and an anti-CD28 antibody can be used.

### Method of Treatment

In one aspect, the present disclosure provides a method of boosting response to or alleviating adverse effects associated with the treatment with a therapeutic anti-TNF alpha monoclonal antibody in a subject in need thereof, comprising an effective amount of the engineered cell described herein.

In some embodiments, the subject suffers a disorder that may benefit from anti-TNF alpha therapy, e.g., a therapy using a therapeutic anti-TNF-alpha monoclonal antibody. Non-limiting examples of disorders that may benefit from an anti-TNF alpha therapy include rheumatoid arthritis (RA), Juvenile idiopathic arthritis (JIA), psoriatic arthritis (PsA), ankylosing spondylitis (AS), adult Crohn's disease (CD), pediatric Crohn's disease, ulcerative colitis (UC), plaque psoriasis (Ps), hidradenitis suppurativa (HS) and uveitis (UV).

In some embodiments, the subject fails to respond to the treatment with a therapeutic anti-TNF alpha monoclonal antibody from the very beginning, losses initial achieved response, or respond adversely. Term “response” as used herein refers to adequate beneficial response of a subject to a treatment. In some embodiments, the therapeutic anti-TNF alpha monoclonal antibody induces ADAs in the subject.

In some embodiments, the engineered cell comprising or expressing a CADAR is derived from T cells isolated from a subject, expanded ex vivo, engineered to comprise a vector for expressing the CADAR, and transfused into the subject. The engineered T cells recognize B cells expressing and presenting ADA-based BCR, wherein the ADA specifically target a therapeutic anti-TNF-alpha monoclonal antibody, and the engineered T cells become activated, resulting in killing of the targeted B cells. In some embodiments, the T cells are autologous cell.

In certain embodiments, the treatment method further comprises administering an agent that increases the efficacy of the engineered cells. For example, a growth factor that promotes the growth and activation of the engineered cells of the present disclosure is administered to the subject either concomitantly with the cells or subsequently to the cells. The growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

In some embodiments, the treatment method further comprises administering an agent that reduces of ameliorates a side effect associated with the administration of the engineered cells. Exemplary side effects include cytokine release syndrome (CRS), and hemophagocytic lymphohistiocytosis (HLH, also termed macrophage activation syndrome (MAS)). The agent administered to treat the side effects can be an agent neutralizing soluble factors such as IFN-gamma, IFN-alpha, IL-2 and IL-6. Such agents include, without limitation, an inhibitor of TNF-alpha (e.g., entanercept) and an inhibitor of IL-6 (e.g., tocilizumab).

Therapeutically effective amounts of the engineered cells can be administered by a number of routes, including parenteral administration, for example, intravenous, intraperitoneal, intramuscular, intrasternal, or intraarticular injection, or infusion.

The engineered cells can be administered in treatment regimens consistent with the immune response to a therapeutic anti-TNF-alpha monoclonal antibody, for example a single or a few doses over one to several days or periodic doses over an extended time. The precise dose to be employed in the formulation will also depend on the route of administration, and the seriousness of the immune response to a therapeutic anti-TNF-alpha monoclonal antibody, and should be decided according to the judgment of the practitioner and each patient's circumstances. The therapeutically effective amount of engineered cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ cells/m2. In a certain embodiment, the dose used in the treatment of human subjects ranges from about 3.8×10⁹ to about 3.8×10¹⁰ cells/m². In additional embodiments, a therapeutically effective amount of the engineered cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of engineered cells is readily determined by one of skill in the art based on the age, weight, sex, and physiological condition of the subject. Effective doses can be extrapolated from dose-response curves derived from in vitro or animal model test systems.

In some embodiments, the engineered cell comprising a CADAR can be administered before, during, following, or in any combination relative to the treatment with a therapeutic anti-TNF alpha monoclonal antibody.

In another aspect, the present disclosure also provides a pharmaceutical composition comprising the engineered cells and a pharmaceutically acceptable diluent and/or carrier. Exemplary diluent and/or carrier include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

TABLE 4
Exemplary sequences of domains
comprised in CADAR

| SEQ | | |
|---|---|---|
| ID | | |
| NO. | Sequence | Domain |
| 20 | MALPVTALLLPLALLLHAARP | CD8 alpha signal |
| | | peptide |
| 21 | IYIWAPLAGTCGVLLLSLVIT | CD8 alpha |
| | | transmembrane |
| | | domain |
| 22 | TTTPAPRPPTPAPTIASQPLSL | CD8 alpha hinge |
| | GRPEACRPAAGAVHTRGLDFAC | region |
| | D | |
| 23 | KRGRKKLLYIFKQPFMRPVQTT | CD137 intracellular |
| | QEEDGCSCRFPEEEEGGCEL | domain |
| 24 | RVKFSRSADAPAYQQGQNQLYN | CD3 zeta (CD247) |
| | ELNLGRREEYDVLDKRRGRDPE | signaling domain |
| | MGGKPRRKNPQEGLYNELQKDK | |
| | MAEAYSEIGMKGERRRGKGHDG | |
| | LYQGLSTATKDTYDALHMQALP | |
| | PR | |
| 25 | GGGGSGGGGSGGGGS | (G4S)₃ |

### EXAMPLE

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1. Expression of CADAR in Human Primary T Cells

A transfer plasmid that includes the DNA sequence encoding a CADAR (see FIG. 2 for schematics of the structure) comprising a scFv derived from adalimumab (scFv-ADL) was designed and synthesized (Genewiz, NJ). The transfer plasmid was then used to generate VSV-G pseudo-typed lentiviral particles using a 4^{th} generation packaging system. In short, 293T cells were transfected at a confluency of 80% with a mixture of the transfer plasmid, the envelope plasmid, the packaging plasmids and Lipofectamine 30000 (Life Technologies). Lentivirus containing supernatant was harvested after 49 hours, filtered through a 0.45 micro PES membrane, concentrated at 1500×g for 45 min at at 4° C. and stored at −80° C.

Human PBMC from healthy donor were activated with CD3/CD28 Dynabeads (Thermo Fisher Scientific) at a 1:1 cell/bead ratio for 24 hrs. 2E+6 T cells were transduced with the lentivirus particles. T cells were cultured in XF T Cell Expansion Medium (STEMCELL Technologies) supplemented with 50 U/ml IL-2 (Thermo Fisher Scientific). Media was changed every 2 to 3 days. D5 after stimulation, positive CADAR-T cells were validated by flow cytometry (Beckman cytoflex).

### Example 2. In Vitro Efficacy Test of CADAR-T Cell

Anti-Adalimumab (ADL) hybridoma cells were generated by immunizing Balb/c mice with purified scFv-ADL protein. B lymphocytes from mouse spleens and myeloma cells were fused. Three rounds of ELISA were used to screen for positive hybridoma clones. One positive (expressing antibodies against ADL) and one negative (not expressing antibodies against ADL) hybridoma cells were cultured in XF T Cell Expansion Medium (STEMCELL Technologies) supplemented with 50 U/ml IL-2 (Thermo Fisher Scientific) and 10% FBS (Gibco). Media was changed every 1 to 2 days.

Positive and negative hybridomas cells were stained first with CFSE (CellTrace, Cat C34554). 1E+4 hybridoma cell/well were stained with CFSE (2.5 μM) for 10 minutes at 37° C., washed twice and resuspended in XF T Cell Expansion Medium (STEMCELL Technologies) supplemented with 50 U/ml IL-2 (Thermo Fisher Scientific) and 10% FBS (Gibco).

CADAR-T cells (8 days after initial activation) and activated T cells without CADAR (mock T) were co-incubated with the stained hybridoma cells for 20 hours at various effector:target (E:T) ratios. Subsequently, cells were spun down at 1,000 rpm for 5 mins at room temperature. Fixable Viability Dye eFluor (eBioscience, Cat 65-0863-18) assay was performed in order to label dead cells. CFSE⁺ Fixable Viability Dye eFluor ⁺hybridoma cell percentage was analyzed by flow cytometry (Beckman, cytoflex). Cytotoxicity of the CARDAR-T cells is calculated based on percent lysis of the hybridoma cells. Killer cytotoxicity (%)=CFSE⁺ Fixable Viability Dye eFluor ⁺hybridoma cells with co-incubated scFv-ADL CADAR (%)−CFSE⁺ Fixable Viability Dye eFluor ⁺hybridoma cells with co-incubated mock T (%). The results of the cytotoxicity assay are shown in Table 5 below. The cytotoxicity of CADAR-T cells increased as E:T ratio increases.

TABLE 5
Killer cytotoxicity (%) of CADAR-T cells

E:T ratio

| | 1:10 | 1:5 | 1:2 | 1:1 |
|---|---|---|---|---|
| CADAR-T | 25.03% | 28.96% | 38.93% | 45.62% |
| Mock-T | 1.14% | 1.21% | 1.51% | 2.48% |

INF-γ production in the co-culture of CADAR-T and hybridoma cells was quantified by ELISA (R&D) after co-culture for 20 hrs. The results are shown in Table 6 below.

TABLE 6
INF-γ production in the co-culture of CADAR-T and hybridoma

| | |
|---|---|
| Positive hybridoma | Negative hybridoma |

| | | | | |
|---|---|---|---|---|
| CADAR-T | 12.8 | ng/ml | 2.77 | ng/ml |
| Mock-T | 0.27 | ng/ml | 0.3 | ng/ml |

### Example 3. In Vivo Efficacy Test of CADAR-T Cell

Positive or negative hybridoma cells are injected intravenously into NSG mice after pre-treatment of mice with intravenous immunoglobulin to minimize FcyR-mediated toxicity against BCR-expressing cells. After a few days, CADAR-T cells (or mock T cells) are injected intravenously. Bioluminescence and/or serum ADA are quantified to monitor CADAR-T cell efficacy. CADAR-T cells control the growth of the positive hybridoma cells but not the negative hybridoma cells, whereas the mock T cells do not control the outgrowth of the positive or negative hybridoma cells.

What is claimed is:

1. An polynucleotide encoding a chimeric anti-drug antibody receptor (CADAR), wherein the chimeric anti-drug antibody receptor comprising an extracellular domain comprising an immunogenic fragment of a therapeutic anti-TNF-alpha monoclonal antibody, a transmembrane domain and an intracellular signaling domain, wherein the immunogenic fragment binds to a B cell receptor (BCR) expressed on a B-cell, wherein a cell expressing the CADAR binds the BCR expressed on the B-cell or induces killing of the B-cell expressing the antibody.

2. The polynucleotide of claim 1, wherein the immunogenic fragment comprises a heavy chain variable region or light chain variable region of the therapeutic anti-TNF-alpha monoclonal antibody, a sequence having at least 90% identify thereof, or a sequence having 1, 2, 3, 4, 5 amino acid residue difference therefrom.

3. The polynucleotide of claim 2, wherein the immunogenic fragment comprises a scFV that comprises the heavy chain variable region and the light chain variable region of the therapeutic anti-TNF-alpha monoclonal antibody, a sequence having at least 90% identify thereof, or a sequence having 1, 2, 3, 4, 5 amino acid residue difference therefrom.

4. The polynucleotide of claim 1, wherein the therapeutic anti-TNF-alpha monoclonal antibody is selected from
(a) adalimumab comprising a heavy chain variable region of SEQ ID NO: 1, and a light chain variable region of SEQ ID NO: 2,
(b) infliximab comprising a heavy chain variable region of SEQ ID NO: 3, and a light chain variable region of SEQ ID NO: 4,
(c) afelimomab comprising a heavy chain variable region of SEQ ID NO: 5, and a light chain variable region of SEQ ID NO: 6,
(d) golimumab comprising a heavy chain variable region of SEQ ID NO: 7, and a light chain variable region of SEQ ID NO: 8, and
(e) certolizumab comprising a heavy chain variable region of SEQ ID NO: 9, and a light chain variable region of SEQ ID NO: 10.

5. The polynucleotide of claim 4, wherein the immunogenic fragment comprises SEQ ID NO: 1, 2, 3, 4, 5, 6, 7, 8, 9 or 10, a sequence having at least 90% identify thereof, or a sequence having 1, 2, 3, 4, 5 amino acid residue difference therefrom.

6. The polynucleotide of claim 4, wherein the immunogenic fragment comprises a scFv comprising:
(a) the heavy chain variable region of SEQ ID NO: 1, and the light chain variable region of SEQ ID NO: 2, a sequence having at least 90% identify thereof, or a sequence having 1, 2, 3, 4, 5 amino acid residue difference therefrom, or
(b) the heavy chain variable region of SEQ ID NO: 3, and the light chain variable region of SEQ ID NO: 4, a sequence having at least 90% identify thereof, or a sequence having 1, 2, 3, 4, 5 amino acid residue difference therefrom, or
(c) the heavy chain variable region of SEQ ID NO: 5, and the light chain variable region of SEQ ID NO: 6, a sequence having at least 90% identify thereof, or a sequence having 1, 2, 3, 4, 5 amino acid residue difference therefrom, or
(d) the heavy chain variable region of SEQ ID NO: 7, and the light chain variable region of SEQ ID NO: 8, a sequence having at least 90% identify thereof, or a sequence having 1, 2, 3, 4, 5 amino acid residue difference therefrom, or
(e) the heavy chain variable region of SEQ ID NO: 9, and the light chain variable region of SEQ ID NO: 10, a sequence having at least 90% identify thereof, or a sequence having 1, 2, 3, 4, 5 amino acid residue difference therefrom.

7. The polynucleotide of claim 4, wherein the therapeutic anti-TNF-alpha monoclonal antibody is adalimumab and the immunogenic fragment comprises
(a) a sequence selected from the group listed in Table 2, or sequence having at least 90% identity thereto, or a sequence having 1, 2, 3, 4, 5 amino acid residue difference therefrom; or
(b) a TNF-alpha binding fragment of adalimumab, or a sequence having at least 90% identify thereof, or a sequence having 1, 2, 3, 4, 5 amino acid residue difference therefrom.

8. The polynucleotide of claim 4, wherein the therapeutic anti-TNF-alpha monoclonal antibody is infliximab and the immunogenic fragment comprises
(a) a sequence selected from the group listed in Table 3 or a sequence having at least 90% identity thereto, or a sequence having 1, 2, 3, 4, 5 amino acid residue difference therefrom; or
(b) a TNF-alpha binding fragment of infliximab, or a sequence having at least 90% identify thereof, or a sequence having 1, 2, 3, 4, 5 amino acid residue difference therefrom.

9. The polynucleotide of claim 7, wherein the TNF-alpha binding fragment is an scFv or a variable region of the corresponding anti-TNF-alpha monoclonal antibody.

10. The polynucleotide of claim 1, wherein the CADAR further comprises a signal peptide domain.

11. The polynucleotide of claim 10, wherein the signal peptide domain is a CD8 alpha signal peptide comprises the sequence of SEQ ID NO: 20 or a sequence having at least 90% identity thereto; or a sequence having 1, 2, 3, 4, 5 amino acid residue difference therefrom.

12. The polynucleotide of claim 1, wherein the transmembrane domain is a transmembrane domain of CD8 alpha.

13. The polynucleotide of claim 12, wherein the transmembrane domain of CD8 alpha comprises the sequence of SEQ ID NO: 21, or a sequence having at least 90% identity thereto; or a sequence having 1, 2, 3, 4, 5 amino acid residue difference therefrom.

14. The polynucleotide of claim 1, wherein the extracellular domain is linked to the transmembrane domain by a hinge region.

15. The polynucleotide of claim 14, wherein the hinge region comprises a hinge region of CD8 alpha.

16. The polynucleotide of claim 15, wherein the hinge region of CD8 alpha comprises the sequence of SEQ ID NO: 22, or a sequence having at least 90% identity thereto; or a sequence having 1, 2, 3, 4, 5 amino acid residue difference therefrom.

17. The polynucleotide of claim 1, wherein the intracellular domain comprises a costimulatory domain and a signaling domain.

18. The polynucleotide of claim 17, wherein the costimulatory domain comprises an intracellular domain of CD137.

19. The polynucleotide of claim 18, wherein the intracellular domain of CD137 comprises the sequence of SEQ ID NO: 23, or a sequence having at least 90% identity thereto; or a sequence having 1, 2, 3, 4, 5 amino acid residue difference therefrom.

20. The polynucleotide of claim 17, wherein the intracellular domain comprises a signaling domain of CD3 zeta.

21. The polynucleotide of claim 20, wherein the signaling domain of CD3 zeta comprises the sequence of SEQ ID NO: 24, or a sequence having at least 90% identity thereto; or a sequence having 1, 2, 3, 4, 5 amino acid residue difference therefrom.

22. A polypeptide encoded by the polynucleotide of claim 1.

23. A vector comprising the polynucleotide of claim 1, wherein the polynucleotide encoding the CADAR is operatively linked to at least one regulatory polynucleotide element for expressing the CADAR.

24. The vector of claim 23, wherein the vector is a plasmid vector, a viral vector, a transposon, a site directed insertion vector, or a suicide expression vector.

25. The vector of claim 23, wherein the vector is a lentiviral vector, a retroviral vector, or an AAV vector.

26. An engineered cell comprising the polynucleotide of claim 1.

27. The engineered cell of claim 26, wherein the engineered cell is a T cell or an NK cell.

28. A method of boosting response to the treatment with a therapeutic anti-TNF alpha monoclonal antibody in a subject in need thereof, comprising administering an effective amount of the engineered cell of claim 26.

29. The method of claim 28, wherein the subject has a condition selected from rheumatoid arthritis (RA), Juvenile idiopathic arthritis (JIA), psoriatic arthritis (PsA), ankylosing spondylitis (AS), adult Crohn's disease (CD), pediatric Crohn's disease, ulcerative colitis (UC), plaque psoriasis (Ps), hidradenitis suppurativa (HS) and uveitis (UV).

30. The method of claim 28, wherein the subject does not respond to or lose initial response to the treatment with the therapeutic anti-TNF alpha monoclonal antibody.

31. The method of claim 28, wherein the therapeutic anti-TNF alpha monoclonal antibody induces anti-drug antibodies in the subject.

32. The method of claim 28, wherein the engineered cell is an autologous cell.

33. The method of claim 28, wherein the engineered cell is an allogeneic cell.

34. The method of claim 28, wherein the method further comprises administering an agent that increases the efficacy of the engineered cells.

35. The method of claim 28, wherein the method further comprises administering an agent that ameliorates a side effect associated with the administration of the engineered cells.

36. The polynucleotide of claim 8, wherein the TNF-alpha binding fragment is an scFv or a variable region of the corresponding anti-TNF-alpha monoclonal antibody.
